(19) United States
(12) Patent Application Publication
(10) Pub. No.: US 2016/0101202 A1
(43) Pub. Date: Apr. 14, 2016

(54) System for Sterilizing Objects Utilizing Germicidal UV-C Radiation and Ozone

(71) Applicant: HEPCO MEDICAL, LLC, Belleair, FL (US)

(72) Inventors: Patricia Carol Gil, Belleair Bluffs, FL (US); Asher Gil, Belleair Bluffs, FL (US); Daniel Gil, Washington, DC (US)

(73) Assignee: HEPCO MEDICAL, LLC, Belleair, FL (US)

(21) Appl. No.: 14/872,387

(22) Filed: Oct. 1, 2015

Related U.S. Application Data
(60) Provisional application No. 62/063,505, filed on Oct. 14, 2014.

(51) Int. Cl. A61L 2/20; A61L 2/10

(52) U.S. Cl. CPC A61L 2/202; A61L 2/10

(57) ABSTRACT

An object sterilization system includes an enclosure having an access door with at least one ultraviolet emitting device supported within the enclosure. The ultraviolet emitting device(s) are for directing ultraviolet radiation on an object placed within the enclosure. A source of power is interfaced to each of the at least one ultraviolet emitting devices, operatively flowing current through each of the at least one ultraviolet emitting devices, thereby each of the at least one ultraviolet emitting device emits ultraviolet radiation for producing ozone and for sterilizing the object.

### CROSS-REFERENCE TO RELATED APPLICATION

This application takes priority from U.S. provisional patent application Ser. No. 62/063,505, filed Oct. 14, 2014. The disclosure of which is hereby incorporated by reference.

### FIELD

This invention relates to the field of disease control and more particularly to a system for reducing pathogens such as bacteria, viruses, fungi, spores, etc., on medical devices and instruments.

### BACKGROUND

Germs/microbes/pathogens, in addition to other chemicals, are left on medical instruments and devices after using such instruments and devices, leading to the spread of such contamination to others if such instruments and devices are used again without killing/eliminating the germs/microbes/pathogens.

Applying chemical products to the instruments/devices kills some germs/microbes/pathogens but does not kill all pathogens, especially those that have a protective shell such as Methicillin-resistant Staphylococcus aureus (MRSA). Liquid sterilizing agents such as oxidizing agents (e.g., hydrogen peroxide and per acetic acid) or aldehydes (e.g., glutaraldehyde and o-phthalaldehyde) are used in some cases to sterilize instruments and devices. Although the use of gas and liquid chemical sterilizing agents and/or high level disinfectants avoids the problem of heat damage (see below), caution must be used to ensure that objects are chemically compatible with the sterilizing agents being used. Using sterilizing agents also requires users to enquire of the manufacturer of the instruments and device to understand specific information regarding compatibility and warranty issues. Further, sterilizing agents typically require users to wear protective masks and gloves. The use of sterilizing agents poses risks and challenges within the workplace environment, especially in the environment of a hospital where some patients are subject to allergic reactions.

In practice today, hospitals attempt to control the spread of germs/microbes/pathogens by either using disposable instruments or devices, or by sterilizing the instruments and devices after each use.

There are many problems that make the use of disposable instruments and devices unattractive, including costs and disposal of contaminated disposable instruments and devices.

Hospitals often use pressurized steam and heat to clean medical instruments and devices. The contaminated instruments and devices are placed in a device known as an autoclave and exposed to high temperatures and pressurized steam for sufficient time as to kill most germs/microbes/pathogens. This method has worked for some germs/microbes/pathogens in the past, but cannot be used on devices that have components with low melting points. Some devices that do not have low melting points are also affected by this process, changing properties of the materials of which they are made after one or more exposures to high temperatures and/or steam. Another setback to this process is the requirement of refilling water supplies that are used to create the steam. Further, as autoclaving uses heat as a way of destroying microorganisms, some microorganisms are not killed even at temperatures of 121 degrees centigrade.

What is needed is a system that will successfully reduce the number of microbes on medical instruments and devices.

### SUMMARY

In one embodiment, an object sterilization system includes an enclosure having an access door with at least one ultraviolet emitting device supported within the enclosure. The ultraviolet emitting device(s) are for directing ultraviolet radiation on an object placed within the enclosure. A source of power is interfaced to each of the at least one ultraviolet emitting devices, operatively flowing current through each of the at least one ultraviolet emitting devices, thereby each of the at least one ultraviolet emitting device emits ultraviolet radiation for sterilizing the object.

In another embodiment, a method of killing pathogens on objects includes providing a device sterilization system comprising an enclosure having at least one opening; the opening having a door; at least one ultraviolet emitting device supported within the enclosure; a shelf for supporting, the shelf passes the ultraviolet radiation there through; a source of electrical current selectively interfaced to each of the ultraviolet emitting devices. The method includes opening the door and placing the object within the enclosure resting on the shelf, then closing the door. Responsive to the closing, the source of electric current provides the electric current to the at least one ultraviolet emitting device; the at least one ultraviolet emitting device thereby emitting ultraviolet radiation and the ultraviolet radiation radiating the object. The ultraviolet radiation breaking oxygen molecules into single oxygen atoms (O1), some of which combine with dioxygen (O2) forming ozone (O3) such that the ultraviolet radiation and the ozone kills at least one pathogens on the object. The method continues with the source of electric current abating the electric current to the at least one ultraviolet emitting device at which time the door is opened and the object removed from the enclosure.

In another embodiment, a object sterilization device includes an enclosure that has an opening for accepting objects; the opening has a door. There is at least one ultraviolet emitting device supported within the enclosure. Each ultraviolet emitting device selectively directs ultraviolet radiation on an object placed in the at least one opening after the door is closed. There is a shelf made of bars for supporting objects. The bars pass the ultraviolet radiation there through. There is also a circuit for controlling a flow of electrical current through each of the ultraviolet emitting devices; the circuit is electrically interfaced to each of the at least one ultraviolet emitting devices. An interlock switch is coupled to the door and electrically coupled to the circuit such that the interlock switch signals the circuit to abate the flow of electrical current through each of the at least one ultraviolet emitting devices when the door is open. A control panel is electrically interfaced to the circuit and has at least one switch for initiating the flow of electrical current through each of the at least one ultraviolet emitting devices.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a perspective view of an exemplary system for reducing the number of pathogens on devices and instruments, shown in an open configuration.

FIG. 2 illustrates a perspective view of an exemplary system for reducing the number of pathogens on devices and instruments, shown in a closed configuration.

FIG. 3 illustrates a front plan cutaway view of the exemplary system for reducing the number of pathogens on devices and instruments.

FIG. 4 illustrates a detail view of the radiating portion of the exemplary system for reducing the number of pathogens on devices and instruments.

FIG. 5 illustrates a schematic view showing an exemplary electrical system of the exemplary system for reducing the number of pathogens on devices and instruments.

FIG. 6 illustrates a schematic cut-away view of the exemplary system for reducing the number of pathogens on devices and instruments showing two shelves.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

Throughout the remainder of this description, the term “pathogen” will be used generically to denote any germ, virus, prion, fungus, spore, microbe, or other pathogen, capable or not capable of infecting a mammal such as a human.

The disclosed system is not limited as to size and, therefore, is anticipated to be capable of being constructed and scaled to any size needed.

A number of microorganisms have a protective hard membrane that protects the cell wall from penetration by UV-C. For such organisms, many of the most deadly ones, it is necessary to first split open this protective membrane in order for the UV-C to penetrate and neutralize the cell. All microorganisms, including endospores such as C.diff and MRSA, have an established level and wavelength of UV that will deactivate them. The sterilization system disclosed herein generates and uses ozone to split open the membrane that protects the cells of such microorganisms.

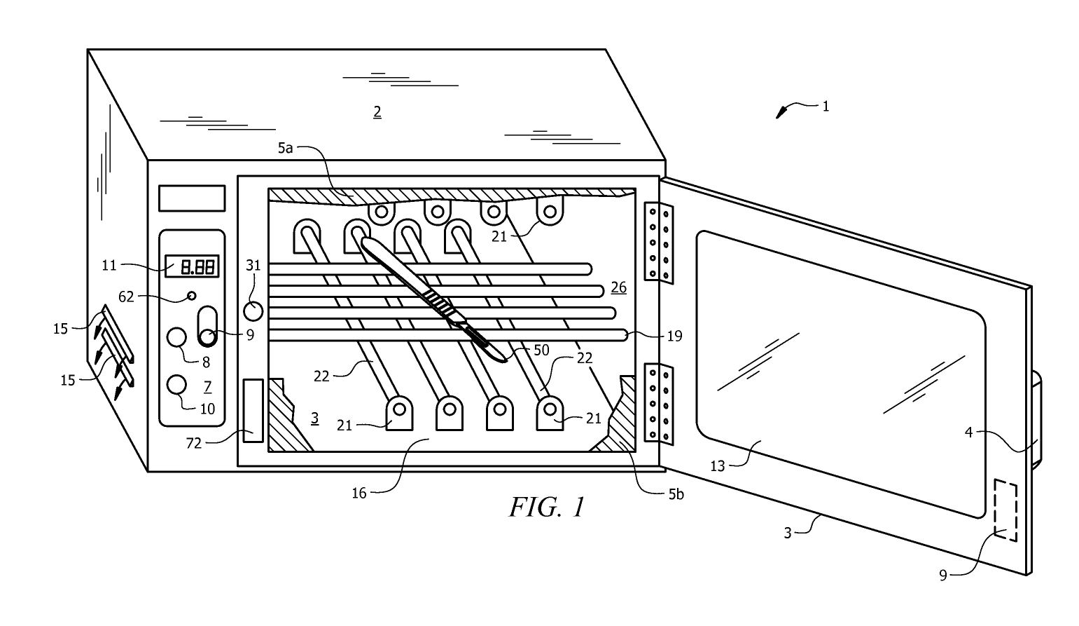

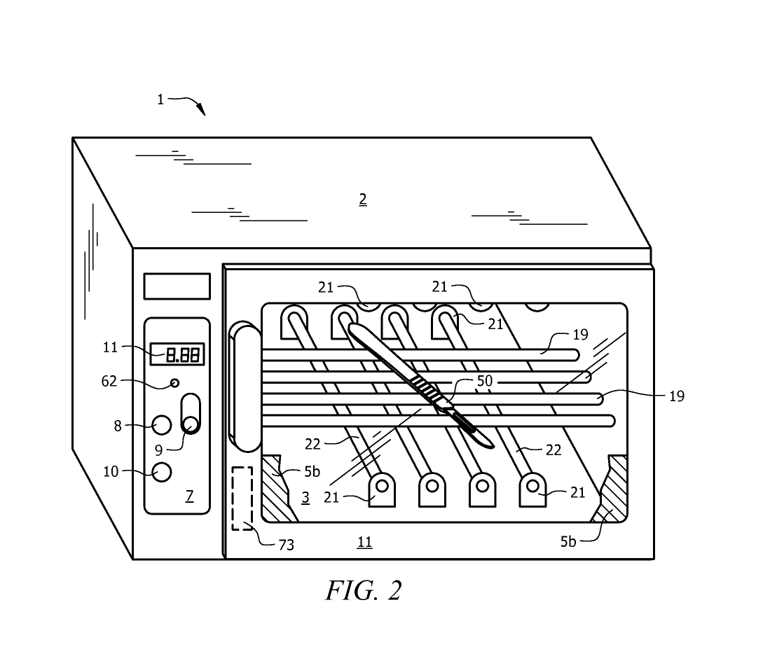

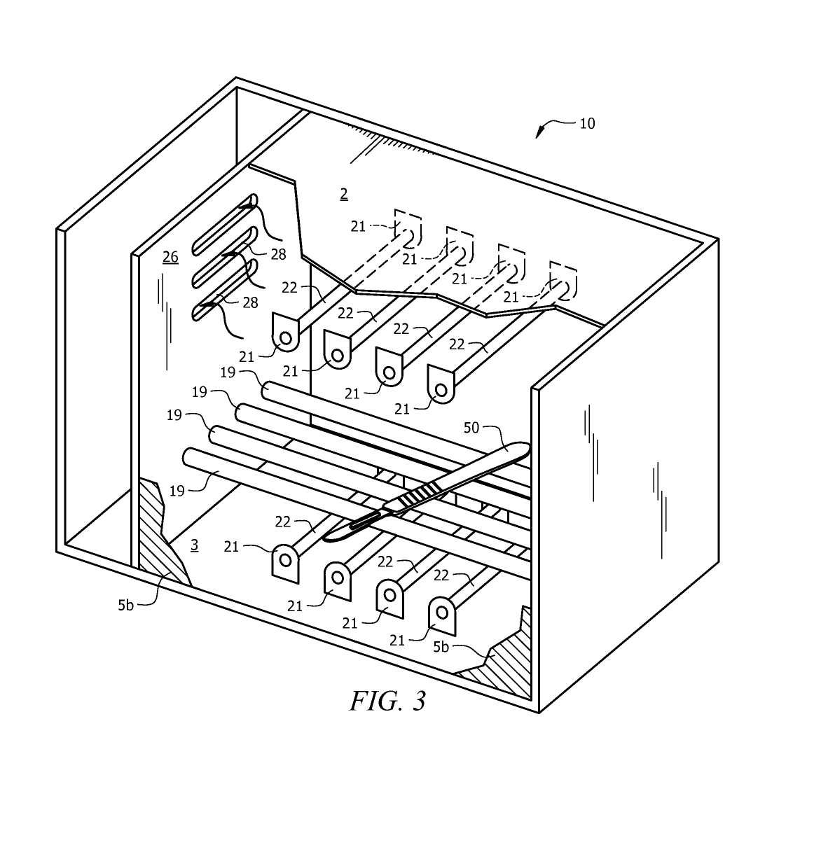

Referring to FIGS. 1-4, view of an exemplary system 1 for reducing the number of pathogens on devices and instruments are shown. The example system 1 is one embodiment of the disclosed invention and there are no limitations as to shape, size, access methods, number of shelves, number of elements, controls, etc.

In the exemplary system 1 for reducing the number of pathogens on devices and instruments, an enclosure 2 has a door 3 to provide access to the inside of the enclosure 2 for placement and removal of devices and instruments, shown as an exemplary scalpel 50 in this example. The door 3 is hinged to the enclosure 2, though any type of access is anticipated. In some embodiments, there is a viewport 13 that is transparent or translucent to allow users to view the contents of the system 1 for reducing the number of pathogens on devices and instruments, though in other embodiments the door 3 is opaque. Being that some wavelengths of ultraviolet radiation is harmful to human beings, especially if viewed by the naked eye, an interlock device is provided including a magnet 9 and a magnetic detector switch 72 (e.g., a reed switch), though any type of interlock is equally anticipated. In operation, when the door 3 is open, the magnet 9 is away from the magnetic detector switch 72, signaling the system 1 to disable emission of ultraviolet radiation. When the door 3 is closed, proximity of the magnet 9 to the magnetic detector switch 72 signals the system 1 to enable emission of ultraviolet radiation based upon various settings that are described later.

It is anticipated that there be a mechanism to prevent the door 3 from inadvertently opening, shown as a magnet 31 that attracts and holds a metal (magnetic attracted material) of the door, or there is a mechanical latch. It is also anticipated that, in some embodiments, there is a handle 4 on the door 3 to facilitate opening of the door 3 for access to the inside of the system 1 for reducing the number of pathogens on devices and instruments.

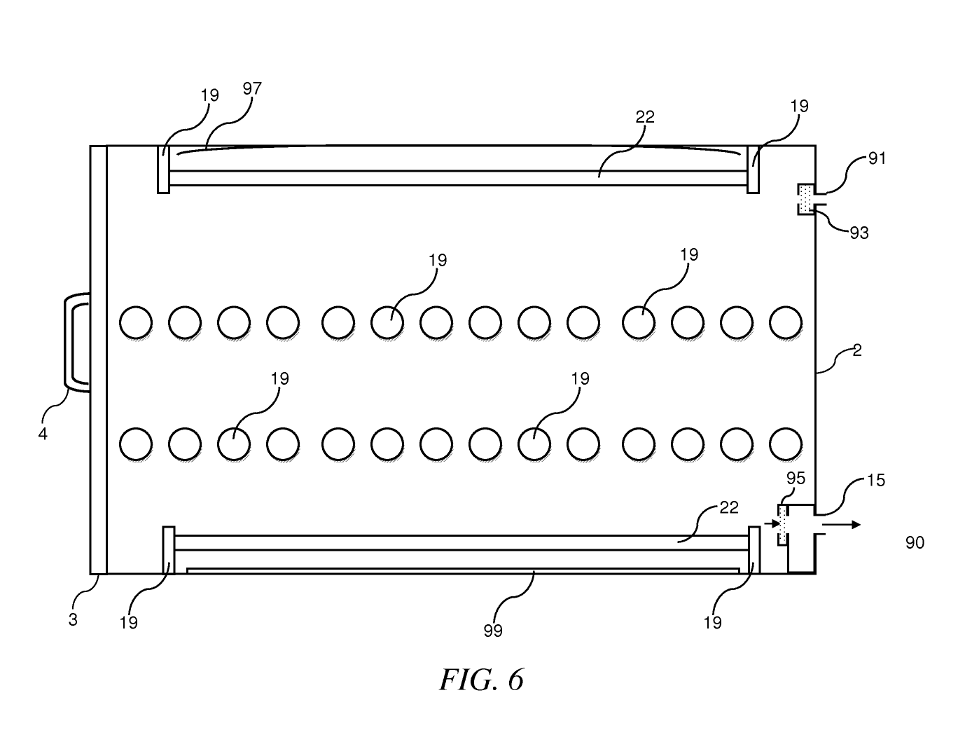

Within the enclosure 2 is at least one radiation emitting device 22 that is/are held by connectors/standoff devices 21. When electrical current flows through the radiation emitting device(s) 22, the radiation emitting device(s) 22 emit ultraviolet radiation directed and/or reflected towards the devices or instruments being sterilized (e.g., scalpel 50). In some embodiments, to protect the radiation emitting device(s) 22, the devices or instruments being sterilized rest upon a shelf made of multiple bars 19. In the example shown, the bars 19 are held in holes in the sides 26 of the enclosure 2, though any mechanism is anticipated for supporting the bars 19. Also, although only one shelf of four bars 19 is shown, any number of shelves having any number of bars 19 is anticipated. For example, refer to FIG. 6 showing two shelves, each having fourteen bars 19. Although it is anticipated that the bars 19 are made of any stiff, supporting material such as glass, plastic, and/or metal, it is preferred that the bars 19 be made of a material that passes ultraviolet radiation so as to fully radiate the devices or instruments being sterilized as the rest on the bars 19. In some embodiments, the bars 19 are made from glass, but glass blocks certain UV wavelengths of radiation. In some embodiments, the bars 19 are made from fused silica or fused quartz. Fused silica or fused quartz have superior transmission of both the ultraviolet and IR spectra radiations. For some applications, the bars 19 are made from other materials or combinations of materials such as ruby, synthetic ruby, and some polymers capable of ultraviolet transmission. Any material that has sufficient structure as to support the intended devices or instruments being sterilized and provides for transmission of the desired radiation is anticipated.

As shown in the examples, the bars 19 are spaced apart from each other by a gap. Although any size gap is anticipated, it is preferred that the gap be small to prevent devices or instruments being sterilized from falling between the bars 19 and hitting the radiation emitting device(s) 22. Although any shelf arrangement that passes ultraviolet radiation in the spectra emitted by the radiation emitting device(s) 22 is anticipated, by making the shelf from bars 19 of ultraviolet passing material, both ultraviolet radiation fully radiates surfaces of the devices or instruments being sterilized and ozone that is generated by the ultraviolet radiation passes through the gaps and surrounds the devices or instruments being sterilized, thereby breaking down the membrane of cells of certain organisms, thereby allowing penetration by the ultraviolet radiation to neutralize the organisms.

As ultraviolet radiation is not visible to the naked human eye, one or more indicators 62 such as lamps or LEDs are provided on a panel 7. In some embodiments, the indicators illuminate with a color or pattern to indicate status of the system 1. For example, the indicator 62 illuminates with a green color to indicate ready/idle, the indicator 62 illuminates with a red color to indicate that the system 1 is operating (in use and emitting ultraviolet radiation), and the indicator 62 illuminates with a color or pattern (e.g. red-blinking) to indicate that the system 1 has malfunctioned (an internal error or a failed radiation emitter 22). In this example, the indicator 62 blinks for a count and then is off for a period, where the count relates to one specific radiation emitter 22 that has failed.

The front panel 7 in this example also has a timer counter 11 that indicates the amount of time remaining in a sterilization cycle and various controls/switches/knobs 8/10 that control the operation of the system 1. The controls initiate operation of the system 1 to initiate a sterilization cycle, manually operate various components such as the vacuum pump 16 and radiation emitting device(s) 22, test the operation of various components and subsystems, etc. In embodiments having a vacuum pump 16, it is anticipated that a vacuum level display 9 is included or there be a control/switch/knob 8/10 that temporary displays vacuum level on the timer display 11. In some embodiments, the controls/switches/knobs select a pre-programmed sterilization cycle from a set of pre-programmed sterilization cycle, typically dependent upon the types of devices or instruments being sterilized. The controls described are examples as it is well known to provided many different mechanisms for controlling radiation emissions and vacuum pumping, all of which are included here within.

Also shown in FIG. 1 are vents 15 for exhaust of internal heat generated by various components and/or for exhausting gases that are evacuated from within the enclosure when the optional vacuum pump is present. Note that it is anticipated that a filter be in line with any gases that are evacuated from within the enclosure 2 should any microorganisms be freed from the devices or objects being sterilized. Although any filter is anticipated, a microbial filter is preferred for this application. Further, in some embodiments, the filter includes a carbon-based filter stage (e.g., charcoal) to reduce emissions of ozone as ozone reacts with carbon to form carbon dioxide (CO₂).

In some embodiments, shields or covers 5a/b5b protect the radiation emitting device(s) 22 and prevent the devices or objects being sterilized from being placed on/near the radiation emitting device(s) 22. In some such embodiments, the shields or covers 5a/b5b are hinged or removable for access, cleaning, and maintenance of the radiation emitting device(s) 22.

In some embodiments, the area housing the electronics and control panel 7 is separated from the sterilization chamber by a wall 26. In some embodiments, one or more vents 28 are provided in the wall 26 for evacuating the sterilization chamber.

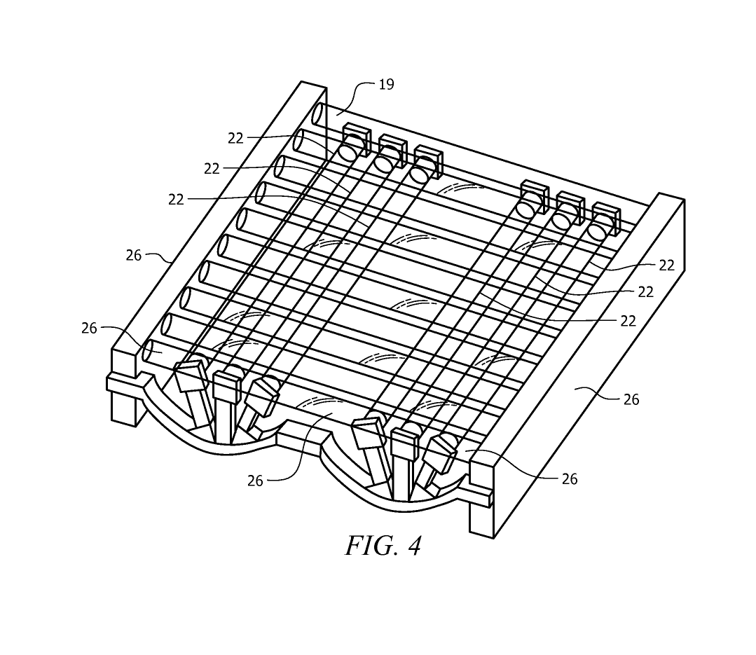

In some embodiments, the radiation emitting device(s) 22 are only positioned or mounted on the floor 3 of the sterilization chamber, while in other embodiments; the radiation emitting device(s) 22 are located in any surface within the sterilization chamber, as for example on an upper surface as shown. There is no limitation as to the number, location, and positioning of the radiation emitting device(s) 22. One exemplary subsystem showing the radiation emitting device(s) 22 in relation to the bars 19 and walls of the enclosure 26 is shown in FIG. 4. Note that it is also anticipated that the ultraviolet radiation be directed towards the devices and instruments being sterilized by any configuration of reflective devices.

Again, because of the potential harmful effects of radiation emanating from the radiation emitting device(s) 22, it is preferred (though not required) to have an interlock system that detects closure of the door 3 tightly against the enclosure 2. There are many known interlock systems that, for example, detecting interlocking of a latching system, etc. In the example shown, a magnet 9 associated with the door 3 interacts with a magnetic switch 72 associated with the enclosure 2, signaling an electrical circuit to enable operation when the magnet 9 is against the magnetic switch 72. Although this is a preferred arrangement, it is also anticipated that the magnet 9 be associated with the enclosure 2 and the magnetic switch 72 be associated with the door 3.

In operation, when contaminated devices and/or instruments are placed atop the support rods 19 and the radiation emitting device(s) 22 are energized, radiation from the radiation emitting devices 22 passes around and through the support rods 19 and radiates the devices and/or instruments. In the preferred embodiment, the support rods 19 are made of a material that attenuate as little of the radiation from the radiation emitting device(s) 22 as possible.

The radiation emitting device(s) 22 emit one or more wavelengths of radiation for the destruction of pathogens. Ultraviolet radiation (400 nm to 100 nm) is categorized into three basic ranges: UVA from 400 nm to 320 nm, UVB from 230 nm to 280 nm, and UVC from 280 nm to 100 nm. For germicidal applications, typically UVB radiation in the range of 280 nm to 240 nm has been shown to be most effective, with 254 nm having the highest efficiency in destroying pathogens.

In some embodiments, the radiation emitting device(s) 22 are ultraviolet emitters or ultraviolet bulbs, often known as UV bulbs or LEDs, emitting radiation with wavelengths of between, for example, 400-100 nm. Such ultraviolet radiation is known to kill at least a subset of known pathogens and, therefore, this radiation is suitable to reduce the number of pathogens on objects placed within the enclosure 2.

Although ultraviolet radiation kills some pathogens and is suitable for that purpose, ultraviolet radiation alone is not effective in killing certain pathogens or classes of pathogens, especially pathogens that have protective envelopes or shells that protect the pathogens from the environment until the pathogens find their way into a suitable environment for growth, such as a wound. An example of such a pathogen is C-diff, which has a protective outer layer and is not significantly affected by UVC radiation. Hydrogen peroxide has been found effective in breaking this outer shell and killing C-diff, but hydrogen peroxide is impractical for use in many scenarios and on many devices and instruments, being dangerous in high concentrations.

Lower wavelengths of ultraviolet radiation will ionize oxygen producing ozone (O₃). For many other applications of ultraviolet radiation, ozone (O₃) production is an unwanted side effect of ultraviolet lamps. For such uses, the ultraviolet lamps are treated or coated to absorb ultraviolet radiation with wavelengths below 254 nm since these lower wavelengths of ultraviolet radiation will ionize oxygen.

Ozone has been found to be effective in killing some pathogens that cannot be effectively killed with ultraviolet radiation alone. Ozone is a strong oxidizing agent that breaks through the encapsulation of some of the more difficult pathogens to kill such as C-diff and MRSA. Ozone is effective in bacterial disinfection and the inactivation of many viruses. Therefore, it is preferred to use a radiation emitting device(s) 22 that emit ultraviolet radiation in approximately the 240-250 nm range and also emit shorter wavelength ultraviolet radiation (e.g. approximately 180 nm) that will produce ozone in the presence of oxygen (O₂).

It is preferred to use radiation emitting device(s) 22 that include emission of ultraviolet radiation in the UVC range and more particularly, in the approximately 180 nm wavelength range to ionize oxygen and purposely create ozone. Such specialized lamps that do not have the surface treatment that filters this wavelength are known and in use in other applications such as water sanitation, often known as germicidal lamps. Such lamps are suitable and anticipated for use as the radiation emitting device(s) 22. These lamps are usually mercury vapor tubes similar to typical fluorescent light bulbs but without any phosphor coating and without any material that impedes the passing of ultraviolet radiation, including ultraviolet radiation in the 253.7 wavelength range which is very good at destroying pathogens. Therefore, these radiation emitting device(s) 22 emit a broader range of ultraviolet that includes the 254 nm wavelength and also shorter wavelengths (e.g. less than 240 nm) that break the bond between dioxygen molecules (O₂+UV−>2O), then the unstable oxygen atoms bond with another dioxygen molecule (O₂+O−>O₃) forming ozone.

Certain wavelengths of ultraviolet radiation are harmful to humans and animals. Exposure to such is known to cause sunburn and eventually skin cancer. Exposure to the naked eye is also known to lead to temporary or permanent vision impairment by damaging the retina of the eye. For this reason, the radiation emitting device(s) 22 is/are shielded within the enclosure 2 and are only illuminated when the door 3 is closed as detected by, for example, sensor 72.

After sufficient exposure to the ultraviolet radiation and/or the ozone, it is desirable to dispose of the ozone. Because ozone is a powerful oxidant, ozone's high oxidizing potential, potentially, causes damage to mucus and respiratory tissues in animals, and also various tissues in plants. Such damage has been observed at concentration levels of about 100 parts per billion. Since ozone reacts with carbon to form carbon dioxide (CO₂), in some embodiments, part or the entire inside surfaces of the enclosure 2 are coated with carbon or carbon granules 99 (see FIG.

6). Since ozone is heavier than air, the ozone will settle towards the bottom of the enclosure 2 and combine with the carbon 99 to form carbon dioxide, which is a harmless gas in low concentrations.

Further, in embodiments having a vacuum pump 16, the vacuum pump 16 pumps gases out of the enclosure 2, for example, before ultraviolet radiation is emitted. In some embodiments, the gases pass through a filter 95 (see FIG. 6) that, optionally, includes carbon (e.g., charcoal).

Although eight independent radiation emitting device(s) 22 are shown (e.g. eight germicidal lamps), any number of radiation emitting device(s) 22 are anticipated including one radiation emitting device 22 and two radiation emitting devices 22. The type of radiation emitting device(s) 22 is not limited in any way to any particular radiation emitting device(s) 22, though known germicidal lamps are shown as examples. It is also anticipated that some subset of the radiation emitting device(s) 22 emit ultraviolet at one wavelength or range of wavelengths and another subset of the radiation emitting device(s) 22 emit ultraviolet at a different wavelength or a different range of wavelengths.

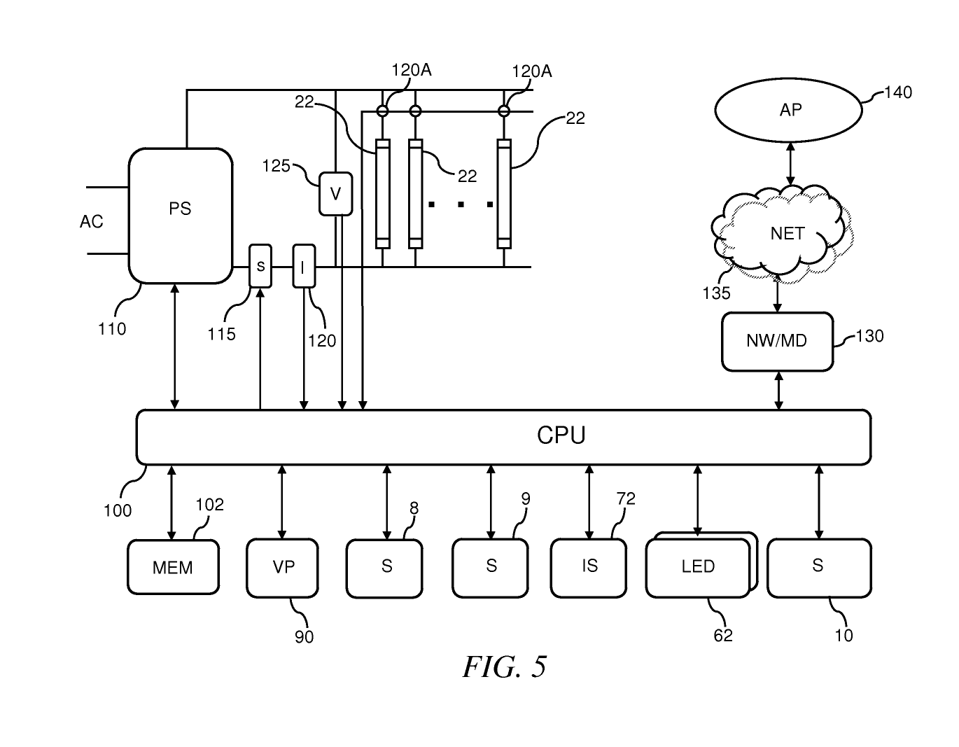

Referring to FIG. 5, block diagram showing an exemplary electrical system of the exemplary system 1 for reducing the number of pathogens on devices and instruments is shown. This is an example of one implementation, utilizing a processor 100 to control operation of the system 1 for reducing the number of pathogens on devices and instruments. There are many other implementations anticipated, with or without the use of a processor 100 or processing element 100, as it is known to implement electrical functionality using discrete electronic components as well.

The exemplary processor-based sub-system is shown having a single processor 100, though any number of processors 100 is anticipated. Many different computer architectures are known that accomplish similar results in a similar fashion and, again, the present invention is not limited in any way to any particular processor 100 or computer system. In this exemplary processor-based sub-system, the processor 100 executes or runs stored programs that are generally stored for execution within a memory 102. The processor 100 is any processor or a group of processors, for example an Intel® 80051 or processors that are known as Programmable Logic Controllers (PLCs). The memory 102 is connected to the processor as known in the industry and the memory 102 is any memory or combination of memory types suitable for operation with the processor 100, such as SRAM, DRAM, SDRAM, RDRAM, DDR, DDR-2, flash, EPROM, EEPROM, etc. The processor 100 is connected to various devices (e.g. sensors, relays, lights, etc.) by any known direct or bus connection.

Although a portable unit powered by batteries and/or solar power is fully anticipated, for AC powered operation, the AC power is conditioned and regulated by a power supply 110, as known in the industry. The power supply 110 provides power for operation of the one or more devices that emit radiation 22, for the processor 100, and for any other component of the processor-based sub-system. In this example, one or more devices that emit radiation 22 are ultraviolet emitting bulbs, similar in operation to small florescent bulbs, though the present invention is not limited to any particular device that emits radiation 22. In general, such devices that emit radiation 22 operate at a specific voltage and draw a typical amount of current per specifications from suppliers of such devices that emit radiation 22. As the devices that emit radiation 22 age or fail, such aging or failure is detected by monitoring of the current and/or voltage provided to devices that emit radiation 22 by one or more sensors 120/125. For example, one sensor 125 monitors voltage over devices that emit radiation 22 and another sensor 120 monitors current to/from devices that emit radiation 22. Outputs of the sensors 120/125 are connected to the processor 100. Upon detection of a failed or aging devices that emit radiation 22, the processor 100 signals such aging or failure by eliminating one or more lamps or LEDs 62, changing the color of one or more lamps or LEDs 62, emitting a sound through a transducer 106, and/or sending a message through the network 135 to, for example, an operations center (computer) 140 that is connected to the network 135. In such, the system 1 includes a network adapter or modem 130 to enable communication through the network 130 to, for example, an operations center 140. This network adapter or modem 130 is any known communications interface, wired or wireless.

Being that it is difficult to discern which of the radiation emitting device(s) 22 has aged or failed because the radiation emitting device(s) 22 don't emit visible light and/or because it is harmful to expose one's eye to the radiation emitted by the radiation emitting device(s) 22, in some embodiments, separate current sensors 120A are configured in series with each of the radiation emitting device(s) 22 and each current sensor 120A is interfaced to the processor 100. In such, the processor 100 reads the current going to/from each of the radiation emitting device(s) 22 to determine, during operation, if the requisite amount of current is flowing through each radiation emitting device(s) 22. When the processor determines one of the radiation emitting device(s) 22 has aged or failed, the processor 100 indicates which of the radiation emitting device(s) 22 has aged or failed by eliminating the lamps/LEDs 62 in a certain pattern, colors, or sequence (e.g., blinking 3 times if the third radiation emitting device 22 has failed) and/or encoding an indication of the failed radiation emitting device(s) 22 in a message that is sent through the network 135 to an operations center 140.

Also, in FIG. 5, one or more switches 8/9 and/or interlock sensors 72 are interfaced to the processor 100. The processor monitors the status of the switch(s) 8/9/72 and enables or disables operation of the radiation emitting device(s) 22 through operation of a power switching device 115 (e.g. solid state switch or relay). In such, it is also anticipated that the processor 100 illuminate one or more lamps or LEDs 62 to signal that the radiation emitting device(s) 22 are operating after proper detection of the interlock switch 72 and applying after applying power to the radiation emitting device(s) 22 through operation of the power switching device 115.

Once the processor 100 detects closure of the interlock switch 72 and/or is controlled by switches 8/9 to initiate operation, the processor 100 closes the power switching device 115, thereby illuminating the radiation emitting device(s) 22 for emission of the ultraviolet radiation onto the enclosure 2. In some embodiments, the processor 100 also illuminates one or more lamps/LEDs 62 to provide feedback to the user that the sterilization process is in operation. In some embodiments, the processor 100 retains power to the ultraviolet emitting bulbs 70 until signaled to stop by, for example, the switches 8/9 or the interlock switch 72. In other embodiments, the processor 100 retains power to the radiation emitting device(s) 22 for a fixed length of time. In either embodiment, once flow of current to the radiation emitting device(s) 22 abates, the lamps/LEDs 62 that were illuminated are extinguished or change color to indicate to the user that the sterilization has stopped.

In some embodiments, the processor terminates the sterilization after a period of time, which is either a fixed time, a selected time, one of a set of fixed times, or algorithmically determined based upon environmental factors such as the type of pathogens that are anticipated, the environment (e.g. pathogens are often more plentiful in warm, humid environments), etc. In some embodiments, it is anticipated that the processor 100 query a remote operations center 140 to obtain information regarding the amount of exposure time, current environmental conditions, pathogen alerts, etc. In some embodiments, the system 1 includes one or more environmental sensors 10, coupled to the processor 100 such as temperature sensors and humidity sensors, etc.

In some embodiments, a vacuum pump 16 is interfaced to the processor 100. The vacuum pump 16 is fluidly interfaced between the interior and exterior of the enclosure 2 such that, upon operation of the vacuum pump 16 under control of the processor 100, the vacuum pump 16 evacuates gases from the interior of the enclosure 2, reducing the pressure within the enclosure 2. As discussed, in some embodiments, the gases are filtered before being exhausted through vents 15. In some embodiments, the filter includes a microbial filter and/or a carbon-based filter (e.g., activated charcoal).

It is noted that the above example includes a processor-based system, but it is well known in the industry to replace the functionality of a processor with discrete components such as timers and sequential logic, etc.

Referring to FIG. 6, a cut-away side view of the enclosure 2 of the system is shown. The cross section of the rods 19 and the length-wise section of the radiation emitting device(s) 22 are visible. The radiation emitting device(s) 22 are shown supported by support device 19, though there is no limitation as to the mounting configuration and removability of each individual radiation emitting device 22. The side of the door 3 and handle 4 are visible.

In some embodiments, one or more reflector(s) 97 are positioned at locations near the radiation emitting device(s) 22 on opposite sides of the radiation emitting device(s) 22 from the devices and instruments being sterilized. When present, the reflector(s) 97 reflect radiation back towards the devices and instruments being sterilized. In some embodiments, the entire interior of the enclosure 2 is a reflective surface to concentrate the ultraviolet radiation on the devices and instruments being sterilized.

Being that ozone (O₃) has more mass than oxygen (O₂) or Nitrogen (N₂) which are the primary gases in our atmosphere, ozone (O₃) produced by the radiation emitting devices 70 tend to gravitate to the bottom strata of the enclosure 2. Although small concentrations of ozone (O₃) is believe to be harmless to plants and animals, as a precautionary step, in some embodiments, a coating or sheet of carbon 99, preferably activated carbon, is located at the bottom of the enclosure. In a preferred alternate embodiment, the sheet of activated carbon 99 is a removable and replaceable fibrous activated carbon mat. This coating or sheet of activated carbon 99 functions similar to a catalytic converter, in which the activated carbon is oxidized by the ozone (O₃), depleting the ozone (O₃) and producing harmless levels of carbon dioxide (CO₂) and carbon monoxide (CO). The coating or sheet of carbon 99 is exposed to ozone (O₃) during each operation of the exemplary system 1. Since the oxidation of the activated carbon in the coating or sheet of carbon 99 depletes layers of carbon, in a preferred embodiment, the coating or sheet of carbon 99 is replaceable with a new coating or sheet of carbon 99.

In some embodiments, a vacuum pump 90 is configured to remove gases from within the enclosure 2, purging the gases through a port/vent 15. In some embodiments, an inlet 91 is provided to slowly replace the evacuated gases with gases (e.g., air) from outside the enclosure 2. In some embodiments, the gases pass through a desiccant 93 before entering the enclosure 2. One example of a desiccant 93 is silica or any other moisture absorbing material. It is anticipated that the cross sectional area of the inlet 91 be small enough so as to slowly allow outside air to enter the enclosure 2. One exemplary operation of the system having such vacuum pump 90 and inlet 91 is, after the door 3 is closed, the vacuum pump 90 is operated until a sensor 10 determines a pre-determined pressure has been achieved (e.g., the pressure within the enclosure 2 is at a pressure less than atmospheric pressure such as 10 pounds per square inch). Once the desired pressure is achieved, outside air continues to slowly flow in through the inlet 91, preferably though a desiccant 93 to remove any humidity from the incoming air.

In some embodiments, the desiccant 93 is replaceable (e.g. in cartridge form) or there is a heating element to evaporate any moisture in the desiccant 93 while the unit is not being used.

Next electrical current is sent through the radiation emitting device(s) 22 and the ultraviolet radiation produces some amount of ozone from oxygen in the incoming air and the ozone and the ultraviolet radiation kills pathogens on the objects placed within the enclosure 2. After a period of time, the electrical current abates and the door 3 is opened to remove the objects from within the enclosure.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. An object sterilization system comprising:
an enclosure having an access door;
at least one ultraviolet emitting device supported within the enclosure, the at least one ultraviolet emitting device for directing ultraviolet radiation on an object placed within the enclosure;
a source of power interfaced to each of the at least one ultraviolet emitting devices, the source of power operatively flowing current through each of the at least one ultraviolet emitting devices, thereby each of the at least one ultraviolet emitting device emits ultraviolet radiation for sterilizing the object.

2. The object sterilization system of claim 1, further comprising a shelf for supporting, the shelf comprising a plurality of bars, the bars pass the ultraviolet radiation there through.

3. The object sterilization system of claim 2, wherein each of the bars is made from a material selected from the group consisting of fused silica and fused quartz.

4. The object sterilization system of claim 1, wherein at least one of the at least one ultraviolet emitting device emits ultraviolet radiation with a wavelength below 240 nm, thereby causing O₂ molecules to split into two O₁ atoms and some of the O₁ atoms combining with other O₂ molecules to form ozone (O₃).

5. The object sterilization system of claim 4, further comprising carbon material at the bottom of the enclosure.

6. The object sterilization system of claim 5, wherein the carbon material is a sheet of activated carbon.

7. The object sterilization system of claim 5, wherein the carbon material is a removable and replaceable sheet of activated carbon.

8. The object sterilization system of claim 4, further comprising a vacuum pump, an input of the vacuum pump interfaced to an area within the enclosure and an output of the vacuum pump interfaced to an area outside of the enclosure such that, during operation of the vacuum pump, gases from within the enclosure are removed and exhausted to the area outside of the enclosure, thereby reducing pressure within the enclosure.

9. The object sterilization system of claim 8, further comprising a filter, the filter in line with the vacuum pump for filtering the gases before the gases are exhausted to the area outside of the enclosure.

10. The object sterilization system of claim 9, wherein the filter, comprises a carbon material of which the ozone (O₃) oxidizes the carbon material producing carbon dioxide from the ozone (O₃).

11. A method of killing pathogens on objects, the method comprising:
providing a device sterilization system comprising:
an enclosure having an opening, the opening having a door;
at least one ultraviolet emitting device supported within the enclosure, the at least one ultraviolet emitting device directing ultraviolet radiation on the object placed in the enclosure;
a shelf for supporting, the shelf comprising a plurality of bars, the bars pass the ultraviolet radiation there through;
a source of electrical current selectively interfaced to each of the at least one ultraviolet emitting device;
opening the door;
placing the object within the enclosure resting on the shelf;
closing the door;
responsive to the closing, the source of electric current providing the electric current to the at least one ultraviolet emitting device, the at least one ultraviolet emitting device thereby emitting ultraviolet radiation, the ultraviolet radiation radiating the object and the ultraviolet radiation breaking oxygen molecules into single oxygen atoms (O₁), some of the single oxygen atoms (O₁) combine with dioxygen (O₂) forming ozone (O₃), the ultraviolet radiation and the ozone killing at least one pathogens on the object;
the source of electric current abating the electric current to the at least one ultraviolet emitting device;
opening the door; and
removing the object from the enclosure.

12. The method of claim 11, further comprising the ozone settling to a bottom of the enclosure and oxidizing an activated carbon material located at the bottom of the enclosure to form carbon monoxide (CO) and/or carbon dioxide (CO2).

13. The method of claim 12, wherein the activated carbon material is a removable and replaceable sheet of activated carbon.

14. The method of claim 11, the device sterilization system further comprising a processor, the processor controlling an amount of time of the step of the source of electric current providing the electric current to the at least one ultraviolet emitting device.

15. The method of claim 14, the device sterilization system further comprising a vacuum pump operatively coupled to the processor, the method further comprising the steps of:
the vacuum pump running to evacuate gases and humidity from the enclosure after the step of closing and before the step of the source of electric current providing the electric current to the at least one ultraviolet emitting device; and
external gases entering through a vent, the gases passing through a desiccant then the gases entering the enclosure.

16. An object sterilization device comprising:
an enclosure having an opening of accepting objects, the opening having a door;
at least one ultraviolet emitting device supported within the enclosure, the at least one ultraviolet emitting device directing ultraviolet radiation on an object placed within the enclosure;
a shelf for supporting objects, the shelf comprising a plurality of bars, the bars pass the ultraviolet radiation there through;
a circuit for controlling a flow of electrical current through each of the at least one ultraviolet emitting devices, the circuit electrically interfaced to each of the at least one ultraviolet emitting devices;
an interlock switch coupled to the door, the interlock switch electrically coupled to the circuit such that the interlock switch signals the circuit to abate the flow of electrical current through each of the at least one ultraviolet emitting devices when the door is open; and
a control panel electrically interfaced to the circuit, the control panel having at least one switch for initiating the flow of electrical current through each of the at least one ultraviolet emitting devices.

17. The object sterilization device of claim 16, wherein at least some of the ultraviolet radiation is at a wavelength below 240 nm which breaks oxygen molecules into single oxygen atom (O₁), and some of the single oxygen atoms combine with dioxygen (O₂) forming ozone (O₃), the ozone for destroying pathogens on the objects, the activated carbon material for oxidizing the ozone to form carbon monoxide (CO) and/or carbon dioxide (CO₂).

18. The object sterilization device of claim 16, wherein each of the bars is made from a material selected from the group consisting of fused silica and fused quartz.

19. The object sterilization device of claim 16, wherein the circuit controls an amount of time for the flow of electrical current through each of the at least one ultraviolet emitting devices.

20. The object sterilization device of claim 16, further comprising a vacuum pump, the circuit controlling the vacuum pump to evacuate gases and humidity from within the enclosure before the flow of electrical current causes the at least one ultraviolet emitting devices to emit ultraviolet radiation.
